(19) United States
(12) Patent Application Publication
(10) Pub. No.: US 2026/0028397 A1
(43) Pub. Date: Jan. 29, 2026

(54) METHODS OF TREATING ANKYLOSING SPONDYLITIS

(71) Applicant: ACELYRIN, INC., Agoura Hills, CA (US)

(72) Inventors: Shao-Lee Lin, Agoura Hills, CA (US); Paul Peloso, Agoura Hills, CA (US)

(21) Appl. No.: 18/854,283

(22) PCT Filed: Apr. 6, 2023
(86) PCT No.: PCT/US2023/065452

§ 371 Date: Oct. 4, 2024

Related U.S. Application Data
(60) Provisional application No. 63/328,533, filed on Apr. 7, 2022.

(51) Int. Cl. C07K 16/24; A61K 39/00

(52) U.S. Cl. CPC C07K 16/244; A61K 2039/505; C07K 2317/31; C07K 2318/00; C07K 2319/31

(57) ABSTRACT

The disclosure relates to novel regimens for treating an inflammatory arthiritis. e.g., psoriatic arthritis. which employ a therapeutically effective amount of an Interleukin- 17 (IL-17) antagonist. e.g., IL-17 binding molecule (e.g., IL-17 antibody or antigen binding fragment thereof. e.g., secukinumab) or IL-17 receptor binding molecule (e.g., IL-17 antibody or antigen binding fragment thereof).

### I. FIELD OF THE INVENTION

The application relates generally to novel dosing regimen for treating an inflammatory arthritis, by administration of a therapeutically effective amount of pharmaceutical composition comprising an engineered bispecific fusion protein, wherein the bispecific fusion protein comprises an IL-17A binding motif and an albumin binding motif.

### II. SEQUENCE LISTING

This application contains a sequence listing in electronic form as an extensible Markup Language (XML) form via the Patent Center and is hereby incorporated by reference in its entirety. The XML-formatted sequence listing, created on Apr. 4, 2023, is named XLRN-002-01WO-ST26.xml, and is 16 KB in size.

### III. BACKGROUND

Ankylosing spondylitis (AS) is a chronic, progressive, inflammatory disease with considerable impact on patient functioning, well-being, and disability. Axial spondyloarthritis (AxSpA) is a type of spondyloarthritis that affects mainly the spine and pelvic joints. In radiographic axial spondyloarthritis (r-AxSpA), the chief symptom is back pain and evidence of fusion on imaging tests. R-AxSpA is chronic, inflammatory disease that impacts the axial skeleton or sacroiliac joints and spine. Non-radiographic axial spondyloarthritis (nr-axSpA) is a type of arthritis in the spine. It causes inflammation, which leads to symptoms like redness, swelling, heat, stiffness, and pain. The condition affects the joints and the entheses, tissues that connect bones to ligaments or tendons.

The prevalence of AS has traditionally been estimated in the range of 0.1-1.9%, with more males affected than females (Sieper et al. Ann Rheum Dis 2001; 60:3-18; Silman & Hochberg Rheum Dis Clin North Am 1996; 22:737-49; Gran & Husby, Semin Arthritis Rheum 1993; 22 (5): 319-34.). Millions of people are affected by ankylosing spondylitis (AS). As a chronic disease of the axial skeleton and large peripheral joints, AS causes inflammatory back pain and stiffness and it is associated with other inflammatory diseases of the skin, eyes and intestines. AS is difficult to diagnose in its early stages and is often an overlooked cause of persistent back pain in young adults. In severe cases, AS may result in complete spinal fusion, causing extreme physical limitation. Thus, there remains a need for a safe and effective treatment for AS.

As the disease progresses, patients with AS experience pain, joint stiffness, and the eventual loss of spinal mobility. These clinical symptoms and subsequent disease progression result in functional limitations and impairment in health-related quality of life (HRQOL) (Dagfinrud et al. Ann Rheum Dis 2004:63:1605-10; Bostan et al. Rheumatol Int 2003; 23:121-6; Zink et al., J Rheumatol 2000; 27:613-22; Ward 1998, Rheum Dis Clin North Am 1998; 24:815-27) and work productivity (Boonen et al. Ann Rheum Dis 2002; 61:429-37; Boonen et al. J Rheumatol 2001; 28:1056-62).

No cure exists for AS. Generally, treatment includes trying to relieve pain and stiffness using medications such as nonsteroidal anti-inflammatory drugs (NSAIDs), corticosteroids, and disease-modifying antirheumatic drugs (DMARDs). In recent years biologic response modifiers that inhibit TNF activity have become established therapies for AS.

### IV. Summary

The invention recognizes that Ankylosing spondylitis (AS) can be treated by compositions that include comprising a bispecific fusion protein, wherein the bispecific fusion protein comprises an IL-17A binding motif and an albumin binding motif. In that manner, the invention provides novel methods for the treatment of AS by administering once a week to a patient a therapeutically effective amount of pharmaceutical composition comprising a bispecific fusion protein, wherein the bispecific fusion protein comprises an IL-17A binding motif and an albumin binding motif. In a related aspect, the invention also provides a bispecific fusion protein for use in the treatment of AS, wherein the bispecific fusion protein comprises an IL-17A binding motif and an albumin binding motif. In another related aspect, the invention provides use of a bispecific fusion protein in the production of a medicament for the treatment of AS, wherein the bispecific fusion protein comprises an IL-17A binding motif and an albumin binding motif.

The interleukin-17 (IL-17) family is a pro-inflammatory cytokine family that contributes to the pathogenesis of several inflammatory diseases. A major source of IL-17 is a lineage of T cells known as T helper 17 cells (Th17 cells), which are distinct from the classical Th1 and Th2 cell subsets. Results of studies in mouse models and in humans have identified a key role of IL-17 and Th17 cells in the pathogenesis of inflammation and autoimmunity as well as in host defense against certain pathogens. Based on these observations, IL-17 and Th 17 cells are considered to be interesting targets for the treatment of several chronic inflammatory diseases such as psoriasis, rheumatoid arthritis (RA), ankylosing spondylitis (AS), systemic lupus erythematosus (SLE) and multiple sclerosis (MS) (Miossec and Kolls, 2012, Nat Rev Drug Discov 11:763-7).

The disulfide-linked homodimeric cytokine IL-17A is a member of the IL-17 family, which also includes IL-17B, IL-17C, IL-17D, IL-17E and IL-17F. Within the family, IL-17A and IL-17F show the highest amino acid sequence homology to each other (50%) and they bind to the same receptors: IL-17 receptor A (IL-17RA) and IL-17 receptor C (IL-17RC). Furthermore, IL-17A can be expressed with IL-17F as a heterodimer. Although IL-17A and IL-17F share high amino acid sequence homology, they perform distinct functions. IL-17A is involved in the development of autoimmunity, inflammation and tumors and also plays important roles in the host defense against bacterial and fungal infections. IL-17F, on the other hand, is mainly involved in mucosal host defense mechanisms (Iwakura et al, 2011, Immunity 34:149-62).

When IL-17A is secreted, it promotes the production of a variety of proinflammatory cytokines, chemokines, antimicrobial peptides and metalloproteinases (MMPs) from fibroblast, endothelial and epithelial cells. One important action of IL-17A is to induce granulopoiesis and neutrophil recruitment to inflammatory sites. However, if uncontrolled, this reaction may lead to chronic inflammation with tissue destruction and neovascularization (Iwakura et al. 2008, Immunol Rev 226:57-79; Reynolds et al. 2010, Cytokine Growth Factor Rev 21:413-23). IL-17A is central in the pathogenesis of psoriasis, a common chronic inflammatory skin disease affecting about 2.5% of the worldwide population (reviewed in Chiricozzi and Krueger, 2013, Expert Opin. Investig. Drugs 22 (8): 993-1005). Studies in patients with RA have shown that IL-17A positive cells are present in the inflamed synovium. In a mouse model of RA, the clinical scores were severely aggravated by administration of II.-17A via intra-articular gene transfer (Lubberts et al. 2002, Inflamm Res 51:102-4). Conversely, inhibition of IL-17A with monoclonal antibodies against the ligand or the receptor protected against development and consequences of arthritis (Lubberts et al. 2004, Arthritis Rheum 50:650-9). In MS patients, the IL-17A gene is reported to be overexpressed (Lock et al. 2002, Nat Med 8:500-8) and IL-17A and Th17 cells have been clearly implicated in the mouse model of experimental autoimmune encephalitis (Cua et al. 2003, Nature 421:744-8; Uyttenhove and Van Snick 2006, Eur J Immunol 36:2868-74). Increased levels of IL-17A have been shown to be clinically correlated with various ocular inflammatory diseases, such as uveitis, scleritis and dry eye disease (DED) in patients suffering from arthritis (Kang et al. 2011, J Korean Med Sci 26:938-44). Recent studies have showed IL-17 and IFNγ positive cells in clinical specimens of coronary atherosclerosis suggesting a local effect on vessel dysfunction (Eid et al. 2009, J Cardiothorac Surg 4:58). Thus, the involvement of IL-17A in several different autoimmune and inflammatory diseases suggests a wide applicability of therapeutics targeting IL-17A.

Targeting of IL-17A or its receptors is the most direct way to block IL-17A-mediated functions. Several biologics that neutralize IL-17A signaling are now in clinical development, including the anti-IL-17A monoclonal antibodies secukinumab and ixekizumab (Patel et al, 2013, Ann Rheum Dis 72 Suppl 2: ii116-23). Secukinumab has been approved for the treatment of psoriasis and is currently investigated for the treatment of psoriatic arthritis (PSA) and AS. Ixekizumab is currently in clinical trials for psoriasis, PsA and RA. Blocking of IL-17 receptor mediated signaling is also under investigation in the clinic, including the human monoclonal anti-IL-17RA antibody brodalumab for treatment of psoriasis, RA and asthma (Hu et al. 2011, Ann N Y Acad Sci 1217:60-76). Thus, clinical efficacy of IL-17A-inhibition has been proven in different diseases, notably in psoriasis, and the safety profile, including phase II and phase III data, shows good tolerability for IL-17A inhibitors (Genovese et al. 2010, Arthritis Rheum 62:929-39 and Hueber et al. 2010, Sci Transl Med 2:52ra72).

Serum albumin is the most abundant protein in mammalian sera (40 g/l; approximately 0.7 mM in humans), and one of its functions is to bind molecules such as lipids and bilirubin (Peters, Advances in Protein Chemistry 37:161, 1985). Serum albumin is devoid of any enzymatic or immunological function. Furthermore, human serum albumin (HSA) is a natural carrier involved in the endogenous transport and delivery of numerous natural as well as therapeutic molecules (Sellers and Koch-Weser, Albumin Structure, Function and Uses, eds Rosenoer et al, Pergamon, Oxford, p 159, 1977). The half life of serum albumin is directly proportional to the size of the animal, where for example human serum albumin has a half life of 19 days and rabbit serum albumin has a half life of about 5 days (McCurdy et al, J Lab Clin Med 143:115, 2004). HSA is widely distributed throughout the body, in particular in the interstitial and blood compartments, where it is mainly involved in the maintenance of osmolarity. Structurally, albumins are single-chain proteins comprising three homologous domains and in total 584 or 585 amino acids (Dugaiczyk et al, Proc Natl Acad Sci USA 79:71, 1982). Albumins contain 17 disulfide bridges and a single reactive thiol, cysteine in position 34, but lack N-linked and O-linked carbohydrate moieties (Peters, 1985, supra; Nicholson et al, Br J Anaesth 85:599, 2000).

Several strategies have been reported to either covalently couple proteins directly to serum albumins or to a peptide or protein that will allow in vivo association to serum albumins. Examples of the latter approach have been described e.g. in WO91/01743, in WO01/45746 and in Dennis et al (J Biol Chem 277:35035-43, 2002). The first document describes inter alia the use of albumin binding peptides or proteins derived from streptococcal protein G (SpG) for increasing the half life of other proteins. The idea is to fuse the bacterially derived, albumin binding peptide/protein to a therapeutically interesting peptide/protein, which has been shown to have a rapid elimination from blood. The thus generated fusion protein binds to serum albumin in vivo, and benefits from its longer half-life, which increases the net half-life of the fused therapeutically interesting peptide/protein. WO01/45746 and Dennis et al relate to the same concept, but here, the authors utilize relatively short peptides to bind serum albumin. The peptides were selected from a phage displayed peptide library. In Dennis et al, earlier work is mentioned in which the enhancement of an immunological response to a recombinant fusion of the albumin binding domain of streptococcal protein G to human complement receptor Type 1 was found. US patent application published as US2004/0001827 (Dennis) also discloses the use of constructs comprising peptide ligands, again identified by phage display technology, which bind to serum albumin and which are conjugated to bioactive compounds for tumor targeting.

Streptococcal protein G (SpG) is a bi-functional receptor present on the surface of certain strains of streptococci and is capable of binding to both IgG and serum albumin (Björck et al, Mol Immunol 24:1113, 1987). The structure is highly repetitive with several structurally and functionally different domains (Guss et al, EMBO J 5:1567, 1986), more precisely three Ig-binding domains and three serum albumin binding domains (Olsson et al, Eur J Biochem 168:319, 1987). The structure of one of the three serum albumin binding domains in SpG has been determined, showing a three-helix bundle fold (Kraulis et al, FEBS Lett 378:190, 1996, Johansson et al, J. Biol. Chem. 277:8114-20, 2002). A 46 amino acid motif was defined as ABD (albumin binding domain) and has subsequently also been designated G148-GA3 (GA for protein G-related albumin binding). In for example WO09/016,043, albumin binding variants of the 46 amino acid motif ABD are disclosed.

Recently, a few T-and B-cell epitopes were experimentally identified within the albumin binding region of Streptococcal protein G strain 148 (G148) (Goetsch et al, Clin Diagn Lab Immunol 10:125-32, 2003). The authors behind the study were interested in utilizing the T-cell epitopes of G148 in vaccines, i.e. to utilize the inherent immune-stimulatory property of the albumin binding region. Goetsch et al additionally found a B-cell epitope, i.e. a region bound by antibodies after immunization, in the sequence of G148.

In pharmaceutical compositions for human administration no immune-response is desired. Therefore, the albumin binding domain G148 is as such unsuitable for use in such compositions due to its abovementioned immune-stimulatory properties.

Furthermore, since tissue penetration rate is negatively associated with the size of the molecule, a relatively large antibody molecule inherently has poor tissue distribution and penetration capacity. Moreover, although antibodies are widely used in a variety of routine contexts owing to high affinity and specificity to a multitude of possible antigens, such as for analytical, purification, diagnostic and therapeutic purposes, they still suffer from several drawbacks. Such drawbacks include the need for complex mammalian expression systems, aggregation tendencies, limited solubility, need to form and stably maintain disulfide bonds, and the risk of unwanted immune responses.

The invention solves these problems using the molecules described here. In an embodiment of the present invention, the IL-17A binding motif includes and/or consists of an amino acid sequence selected from:

i)
(SEQ ID NO. 1)
EX₂DX₄AX₆X₇EIX₁₀X₁₁LPNLX₁₆X₁₇X₁₈QX₂₀X₂₁AFIX₂₅X₂₆LX₂₈X₂₉

wherein, independently from each other,
X₂ is selected from A, H, M and Y;
X₄ is selected from A, D, E, F, K, L, M, N, Q, R, S and Y;
X₆ is selected from A, Q and W;
X₇is selected from F, I, L, M, V, W and Y;
X₁₀ is selected from A and W;
X₁₁ is selected from A, D, E, F, G, L, M, N, Q, S, T and Y;
X₁₆ is selected from N and T;
X₁₇ is selected from H, W and Y;
X₁₈ is selected from A, D, E, H and V;
X₂₀ is selected from A, G, Q, S and W;
X₂₁ is selected from A, D, E, F, H, K, N, R, T, V, W and Y;
X₂₅ is selected from A, D, E, G, H, I, L, M, N, Q, R, S, T and V;
X₂₆ is selected from K and S;
X₂₈ is selected from I, L, N and R; and
X₂₉ is selected from D and R;
and
ii) an amino acid sequence which has at least 89% identity to the sequence defined in i).

As the skilled person will realize, the function of any polypeptide, such as the IL-17A binding capacity of the polypeptide of the present disclosure, is dependent on the tertiary structure of the polypeptide. It is therefore possible to make minor changes to the sequence of amino acids in a polypeptide without affecting the function thereof. Thus, the disclosure encompasses modified variants of the IL-17A binding polypeptide, which are such that the IL-17A binding characteristics are retained.

In another embodiment of the invention, the fusion protein or conjugate of this second aspect comprises two monomers of the IL-17A binding polypeptide of the first aspect, whose amino acid sequences may be the same or different, linked by an albumin binding moiety. In a specific embodiment of this construct, the fusion protein or conjugate comprises two IL-17A binding monomers with an albumin binding moiety between them. Said albumin binding moiety may e.g. be a “GA” albumin binding domain from streptococcal protein G, such as “GA3”, or a derivative thereof as described in any one of WO2009/016043, WO2012/004384, WO2014/048977 and WO2015/091957.

In another embodiment, the albumin binding motif consists of an amino acid sequence LAX₃AKX₆X₇ANX₁₀ELDX₄YGVSDFYKRLIX₂₆KAKTVEGVEALKX₃₉X₄₀ILX₄₃X₄₄LP (SEQ ID. No. 2), wherein independently of each other
X₃ is selected from E, S, Q and C;
X₆ is selected from E, S and C;
X₇is A;
X₁₀ is selected from A, S and R;
X₁₄ is selected from A, S, C and K;
X₂₆ is selected from D and E;
X₃₉ is selected from D and E;
X₄₀is A;
X₄₃ is selected from A and K;
X₄₄ is selected from A and S; and
P in position 46 is present or absent.

In one embodiment of the invention, the bispecific fusion protein is izokibep. Izokibep may also be referred to as ABY-035 or IMG-020.

Izokibep is a small protein therapeutic designed to inhibit interleukin-17A (IL-17A) with higher potency and the potential for greater tissue penetration due to its markedly smaller size when compared to traditional monoclonal antibodies.

Izokibep has enhanced potency as it blocks the homodimeric IL-17A target protein by binding to both sub-units simultaneously with a very high affinity. In certain embodiments, Kp for izokibep binding to IL-17A is as low as 0.3 pM. Klint et al. Izokibep-Preclinical Development and First-in-Human Study of a Novel IL-17A Neutralizing Affibody Molecule in Patients with Plaque Psoriasis. mAbs. 2023 (manuscript accepted, in publication). In particular, the two IL-17A binding domains bind to the dimeric IL-17A homodimers at the same time, and the two IL17A binding domains are connected by albumin binding domain.

Advantageously, the presence of albumin binding domain increases the half-life of izokibep. The half-life of izokibep may be a few days. In certain embodiments, the half-life of izokibep is from about 5 to about 20 days. In certain embodiments, the half-life of izokibep is from about 10 to about 15 days. In certain embodiments, the half-life of izokibep is about 12 days. Importantly, as a result of enhanced half-life of izokibep, it can engage with the pharmacological targets for longer duration. Izokibep also has a well-established safety profile. In certain embodiments of the invention, izokibep is safe for patients up to 3 years without any observed increased risk of infection or any significant increase in anti-drug antibodies (ADAs). The presence of or a significant increase in ADAs can impact exposure of the drug and/or the clinical response of the drug in the patients.

In contemporary treatments for HS, exposures of the drugs are lower compared to other inflammatory conditions. Advantageously, the high potency of izokibep to IL-17A, as well as the small molecular size of izokibep, leads to improved tissue penetration and target engagement and therefore provide the potential for differentiated clinically meaningful benefit for patients. In certain embodiments, the size of izokibep is one-tenth ( 1/10^{th}) of those of typical monoclonal IL-17A antibodies. In certain embodiments, izokibep has quick and therapeutic effects in patients suffering from HS and Psoriatic Arthritis.

In certain embodiments, the bispecific fusion protein is SEQ ID. NO. 3.

In certain embodiments, the bispecific fusion protein sequence comprises the peptide described in the amino acid sequence below or a fragment thereof:

AEAKYAKEADDAAVEIASLPNLTWDQWYAFIQKLRDDPSQSSELLSEAK
KLNDSQAPKASGSLAEAKEAANAELDSYGVSDFYKRLIDKAKTVEGVEA
LKDAILAALPGTGGGGSAEAKYAKEADDAAVEIASLPNLTWDQWYAFIQ
KLRDDPSQSSELLSEAKKLNDSQAPK

In another embodiment of the invention, a pharmaceutical composition comprising a therapeutically effective amount of the bispecific fusion protein, preferably izokibep, is administered to a patient suffering from ankylosing spondylitis.

Another embodiment of the invention provides a pharmaceutical composition of the bispecific fusion protein, preferably izokibep. Preferably, the pharmaceutical composition is an injectable solution. In another embodiment of the invention, the bispecific fusion protein, preferably izokibep, is administered to the patient subcutaneously.

In another embodiment of the invention, the pharmaceutical composition comprises 20-200 mg izokibep. In another embodiment of the invention, the pharmaceutical composition comprises 40 mg izokibep. In another embodiment of the invention, the pharmaceutical composition comprises 80 mg izokibep. In another embodiment of the invention, the pharmaceutical composition comprises 160 mg izokibep.

In another embodiment of the invention, the pharmaceutical composition comprises at least one additional excipient.

In another embodiment of the invention, a pharmaceutical composition comprising a therapeutically effective amount of the bispecific fusion protein, preferably izokibep, is administered to a patient suffering from ankylosing spondylitis once a week.

In another embodiment of the invention, a pharmaceutical composition comprising 40 mg izokibep, is administered to a patient suffering from ankylosing spondylitis once a week.

In another embodiment of the invention, a pharmaceutical composition comprising 40 mg izokibep, is administered to a patient suffering from ankylosing spondylitis once every two weeks.

In another embodiment of the invention, a pharmaceutical composition comprising 80 mg izokibep, is administered to a patient suffering from ankylosing spondylitis once every two weeks.

In another embodiment of the invention, a pharmaceutical composition comprising 160 mg izokibep, is administered to a patient suffering from ankylosing spondylitis once a week.

In another embodiment of the invention, a pharmaceutical composition comprising 160 mg izokibep, is administered to a patient suffering from ankylosing spondylitis once every two weeks.

In another embodiment of the invention, a pharmaceutical composition comprising 160 mg izokibep, is administered to a patient suffering from ankylosing spondylitis once every four weeks.

### V. Detailed Description

The present invention is directed to a novel method for the treatment of AS by administering once a week to a patient a therapeutically effective amount of pharmaceutical composition comprising a bispecific fusion protein, wherein the bispecific fusion protein comprises an IL-17A binding motif and an albumin binding motif.

A preferred example of the bispecific fusion protein is izokibep (ABY-035 or IMG-020), IL-17 inhibitor, is currently in clinical trials for treatment of AS (Rondon et al., Adv. Funct. Mater., 2021, 31, 2101633-2101665). Izokibep is a bispecific fusion protein comprising two units of an engineered variant of protein Z derived from the B domain of staphylococcal Protein A and structured as a triple a-helix bundle, with a high affinity for interleukin-17A (IL-17A) and one ABD domain (5 kDa) with high affinity for SA.

In certain embodiments, the bispecific fusion protein is SEQ ID. NO. 3.

In certain embodiments, the bispecific fusion protein sequence comprises the peptide described in the amino acid sequence below or a fragment thereof:

AEAKYAKEADDAAVEIASLPNLTWDQWYAFIQKLRDDPSQSSELLSEAK
KLNDSQAPKASGSLAEAKEAANAELDSYGVSDFYKRLIDKAKTVEGVEA
LKDAILAALPGTGGGGSAEAKYAKEADDAAVEIASLPNLTWDQWYAFIQ
KLRDDPSQSSELLSEAKKLNDSQAPK

### IL-17A Binding Motif

The invention discloses IL-17A binding motifs, which could for example be used for diagnostic, prognostic and therapeutic applications. It is an object of the present disclosure to provide IL-17A binding motifs, which may be used as domains in fusion proteins comprising one or more additional domains having similar or other functions.

These and other objects which are evident to the skilled person from the present disclosure are met by different aspects of the invention as claimed in the appended claims and as generally disclosed herein.

Thus, in the first aspect of the disclosure, the IL-17A binding motif consists of an amino acid sequence selected from (SEQ ID NO: 1):

i)
EX₂DX₄AX₆X₇EIX₁₀X₁₁LPNLX₁₆X₁₇X₁₈QX₂₀X₂₁AFIX₂₅X₂₆LX₂₈X₂₉

wherein, independently from each other,
X₂ is selected from A, H, M and Y;
X₄ is selected from A, D, E, F, K, L, M, N, Q, R, S and Y;
X₆ is selected from A, Q and W;
X₇is selected from F, I, L, M, V, W and Y;
X₁₀ is selected from A and W;
X₁₁ is selected from A, D, E, F, G, L, M, N, Q, S, T and Y;
X₁₆ is selected from N and T;
X₁₇ is selected from H, W and Y;
X₁₈ is selected from A, D, E, H and V;
X₂₀ is selected from A, G, Q, S and W;
X₂₁ is selected from A, D, E, F, H, K, N, R, T, V, W and Y;
X₂₅ is selected from A, D, E, G, H, I, L, M, N, Q, R, S, T and V;
X₂₆ is selected from K and S;
X₂₈ is selected from I, L, N and R; and
X₂₉ is selected from D and R;
and
ii) an amino acid sequence which has at least 89% identity to the sequence defined in i).

The above definition of a class of sequence related, IL-17A binding polypeptides is based on a statistical analysis of a number of random polypeptide variants of a parent scaffold, that were selected for their interaction with IL-17A in several different selection experiments. The identified IL-17A binding motif, or “BM”, corresponds to the target binding region of the parent scaffold, which region constitutes two alpha helices within a three-helical bundle protein domain. In the parent scaffold, the varied amino acid residues of the two BM helices constitute a binding surface for interaction with the constant Fc part of antibodies. In the present disclosure, the random variation of binding surface residues and subsequent selection of variants have replaced the Fc interaction capacity with a capacity for interaction with IL-17A.

As the skilled person will realize, the function of any polypeptide, such as the IL-17A binding capacity of the polypeptide of the present disclosure, is dependent on the tertiary structure of the polypeptide. It is therefore possible to make minor changes to the sequence of amino acids in a polypeptide without affecting the function thereof. Thus, the disclosure encompasses modified variants of the IL-17A binding polypeptide, which are such that the IL-17A binding characteristics are retained.

In this way, also encompassed by the present disclosure is an IL-17A binding polypeptide comprising an amino acid sequence with 89% or greater identity to a polypeptide as defined in i). In some embodiments, the polypeptide may comprise a sequence which is at least 93%, such as at least 96% identical to a polypeptide as defined in i). For example, it is possible that an amino acid residue belonging to a certain functional grouping of amino acid residues (e.g. hydrophobic, hydrophilic, polar etc) could be exchanged for another amino acid residue from the same functional group.

In some embodiments, such changes may be made in any position of the sequence of the IL-17A binding polypeptide as disclosed herein. In other embodiments, such changes may be made only in the non-variable positions, also denoted scaffold amino acid residues. In such cases, changes are not allowed in the variable positions, i.e. positions denoted with an “X” in sequence i).

The term “% identity”, as used throughout the specification, may for example be calculated as follows. The query sequence is aligned to the target sequence using the CLUSTAL W algorithm (Thompson et al, Nucleic Acids Research, 22:4673-4680 (1994)). A comparison is made over the window corresponding to the shortest of the aligned sequences. The shortest of the aligned sequences may in some instances be the target sequence. In other instances, the query sequence may constitute the shortest of the aligned sequences. The amino acid residues at each position are compared and the percentage of positions in the query sequence that have identical correspondences in the target sequence is reported as % identity.

In one particular embodiment, there is provided a polypeptide as defined above, wherein, in sequence i),
X₂ is selected from A, H and M;
X₄ is selected from A, D, E, F, L, M, N, Q, R and Y;
X₁₁ is selected from A, D, E, F, G, L, M, N, S, T and Y;
X₁₈ is selected from A, D, E and V;
X₂₀ is selected from A, G, Q and W;
X₂₁ is selected from E, F, H, N, R, T, V, W and Y;
X₂₅ is selected from A, D, E, G, H, I, L, N, Q, R, S, T and V; and
X₂₈ is selected from I, N and R.

In another particular embodiment, there is provided a polypeptide as defined in the paragraph immediately above, wherein in addition, in sequence i),
X₁₆ is T;
X₁₇ is W,
X₂₁ is selected from E, F, H, W, T and Y;
X₂₅ is selected from A, D, E, G, H, I, L, N, Q, R, S and T;
X₂₆ is K; and
X₂₉is D.
“Xₙ” and “Xₘ” are used herein to indicate amino acids in positions n and m in the sequence i) as defined above, wherein n and m are integers indicating the position of an amino acid within sequence i) as counted from the N terminus. For example, X₃ and X₇ indicate the amino acids in positions three and seven, respectively, from the N-terminal end of sequence i).

In certain embodiments of the invention, there are provided polypeptides wherein Xₙ in sequence i) is independently selected from a group of possible residues as listed in Table 1. The skilled person will appreciate that Xₙ may be selected from any one of the listed groups of possible residues and that this selection is independent from the selection of amino acids in Xₘ, wherein n≠m. Thus, any of the listed possible residues in position Xₙ in Table 1 may be independently combined with any of the listed possible residues any other variable position in Table 1.

TABLE 1

| | |
|---|---|
| X₂ | A, M, Y |
| X₂ | A, M |
| X₄ | A, D, E, F, K, L, M, N, Q, R, Y |
| X₄ | A, D, E, F, L, M, N, Q, R, S, Y |
| X₄ | A, D, E, F, L, M, N, Q, R, Y |
| X₄ | A, D, E, F, L, N, Q, R, Y |
| X₄ | A, D, E, F, M, N, Q, Y |
| X₄ | A, D, E, F, L, Q, R |
| X₄ | A, D, E, L, Q, R |
| X₄ | A, D, Q, R |
| X₄ | D, Q |
| X₄ | A, D, E, F, N, Q, Y |
| X₄ | D, E, F, N, Q, R, Y |
| X₄ | D, E, N, Q, Y |
| X₄ | D, E, N, Y |
| X₄ | D, E, Q, Y |
| X₄ | D, E, Y |
| X₄ | D, E |
| X₄ | D |
| X₄ | Q |
| X₆ | A, Q |
| X₆ | A, W |
| X₆ | Q, W |
| X₆ | W |
| X₆ | A |
| X₆ | Q |
| X₇ | F, I, L, V, W, Y |
| X₇ | F, I, L, V, Y |
| X₇ | I, L, V, Y |
| X₇ | L, V, Y |
| X₇ | L, V |
| X₇ | F, M, V, W, Y |
| X₇ | F, V, W, Y |
| X₇ | F, V, W |
| X₇ | F, V |
| X₇ | V |
| X₇ | L |
| X₇ | Y |
| X₁₀ | A |
| X₁₀ | W |
| X₁₁ | A, D, E, F, G, L, M, N, S, T, Y |
| X₁₁ | A, D, E, F, L, M, S, T |
| X₁₁ | A, D, E, F, G, L, M, N, S, Y |
| X₁₁ | A, D, E, F, G, S, Y |
| X₁₁ | A, D, E, F, L, M, S |
| X₁₁ | A, D, E, L, M, S |
| X₁₁ | A, D, E, L, S |
| X₁₁ | A, D, E, L, M |
| X₁₁ | A, D, E, L |
| X₁₁ | D, E, L |
| X₁₁ | D, L |
| X₁₁ | A, D, E, F, S, Y |
| X₁₁ | A, D, E, S |
| X₁₁ | A, D, S |
| X₁₁ | A, S |
| X₁₁ | A, D |
| X₁₁ | D, S |
| X₁₁ | L |
| X₁₁ | D |
| X₁₁ | S |
| X₁₁ | A |
| X₁₁ | E |
| X₁₆ | T |
| X₁₆ | N |
| X₁₇ | H, W |
| X₁₇ | Y, W |
| X₁₇ | H, Y |
| X₁₇ | W |
| X₁₇ | H |
| X₁₇ | Y |
| X₁₈ | A, D, E, V |
| X₁₈ | A, D, E, H |
| X₁₈ | A, D, H |
| X₁₈ | A, D, E |
| X₁₈ | A, D |
| X₁₈ | D, E |
| X₁₈ | D |
| X₁₈ | A |
| X₂₀ | A, G, Q, W |
| X₂₀ | A, Q, S, W |
| X₂₀ | A, Q, W |
| X₂₀ | G, Q, W |
| X₂₀ | G, W |
| X₂₀ | Q, W |
| X₂₀ | A, W |
| X₂₀ | W |
| X₂₀ | A |
| X₂₀ | Q |
| X₂₁ | A, D, E, F, H, N, R, T, V, W, Y |
| X₂₁ | A, D, E, F, H, N, R, V, W, Y |
| X₂₁ | E, F, H, N, R, V, W, Y |
| X₂₁ | F, H, N, R, V, W, Y |
| X₂₁ | E, F, H, T, W, Y |
| X₂₁ | E, F, H, W, Y |
| X₂₁ | F, H, R, W, Y |
| X₂₁ | F, H, W, Y |
| X₂₁ | F, W, Y |
| X₂₁ | W, Y |
| X₂₁ | F, Y |
| X₂₁ | F, W |
| X₂₁ | Y |
| X₂₁ | W |
| X₂₁ | F |
| X₂₅ | A, D, E, G, H, I, L, N, Q, R, S, T, V |
| X₂₅ | A, D, E, G, H, I, L, N, Q, R, S, T |
| X₂₅ | A, D, E, G, H, N, Q, R, S, T, V |
| X₂₅ | D, E, G, N, Q, R, S, T, V |
| X₂₅ | D, E, N, Q, R, V |
| X₂₅ | A, D, E, G, I, L, N, Q, R, S, T |
| X₂₅ | D, E, G, N, Q, R, S |
| X₂₅ | D, E, N, Q, R, S |
| X₂₅ | A, E, G, L, N, Q, R, S, T |
| X₂₅ | E, N, Q, R, S |
| X₂₅ | E, N, Q, S, T |
| X₂₅ | E, N, Q, S |
| X₂₅ | N, Q, R |
| X₂₅ | E, Q, R |
| X₂₅ | Q, R, S |
| X₂₅ | Q, S |
| X₂₅ | Q, R |
| X₂₅ | Q |
| X₂₅ | S |
| X₂₅ | N |
| X₂₅ | E |
| X₂₆ | K |
| X₂₆ | S |
| X₂₈ | I, N, R |
| X₂₈ | I, L, R |
| X₂₈ | I, R |
| X₂₈ | N, R |
| X₂₈ | I, N |
| X₂₈ | R |
| X₂₈ | I |
| X₂₈ | N |
| X₂₉ | D |
| X₂₉ | R |

In a more specific embodiment, defining a sub-class of IL-17A binding polypeptides, sequence i) fulfills at least six of the eleven conditions I-XI:
I. X₂ is A;
II. X₄ is selected from D, E and Q;
III. X₆ is A;
IV. X₇is selected from F and V;
V. X₁₆ is T;
VI. X₁₇is W;
VII. X₁₈ is selected from A and D;
VIII. X₂₀ is W;
IX. X₂₆ is K;
X. X₂₈ is R; and
XI. X₂₉ is D.

In some examples of an IL-17A binding polypeptide of the invention, sequence i) fulfills at least seven of the eleven conditions I-XI. More specifically, sequence i) may fulfill at least eight of the eleven conditions I-XI, such as at least nine of the eleven conditions I-XI, such as at least ten of the eleven conditions I-XI, such as all of the eleven conditions I-XI.

In some embodiments, for the IL-17A binding polypeptide, X₂X₆, X₂X₁₀ or X₆X₁₀ are independently AA. In some embodiments, X₂X₁₇, X₂X₂₀, X₆X₁₇, X₆X₂₀, X₁₀X₁₇ or X₁₀X₂₀ are independently AW. In some embodiments, X₂X₂₈, X₆X₂₈ or X₁₀X₂₈ is AR. In some embodiments, X₁₇X₂₈ or X₂₀X₂₈ is WR. In some embodiments, X₁₇X₂₀ is WW.

In one embodiment, the sequences of individual IL-17A binding motifs correspond to amino acid positions 8-36 in SEQ ID NO:1-1216 presented in U.S. Pat. No. 10,934,335, which is incorporated by reference in its entirety.

In one embodiment of the IL-17A binding polypeptide, sequence i) corresponds to the sequence from position 8 to position 36 in a sequence selected from the group consisting of SEQ ID NO: 1-1216 presented in U.S. Pat. No. 10,934,335, which is incorporated by reference in its entirety.

In one embodiment, sequence i) corresponds to the sequence from position 8 to position 36 in a sequence selected from the group consisting of SEQ ID NO:1-66, 1200, 1206 and 1214, presented in U.S. Pat. No. 10,934,335, which is incorporated by reference in its entirety.

In one embodiment, sequence i) corresponds to the sequence from position 8 to position 36 in a sequence selected from the group consisting of SEQ ID NO: 1-66 presented in U.S. Pat. No. 10,934,335, which is incorporated by reference in its entirety.

In one embodiment, sequence i) corresponds to the sequence from position 8 to position 36 in a sequence selected from the group consisting of SEQ ID NO: 1-35 presented in U.S. Pat. No. 10,934,335, which is incorporated by reference in its entirety. In one embodiment, sequence i) corresponds to the sequence from position 8 to position 36 in a sequence selected from the group consisting of SEQ ID NO:1-27 presented in U.S. Pat. No. 10,934,335, which is incorporated by reference in its entirety. In one embodiment, sequence i) corresponds to the sequence from position 8 to position 36 in a sequence selected from the group consisting of SEQ ID NO: 1-10 presented in

U.S. Pat. No. 10,934,335, which is incorporated by reference in its entirety. In one embodiment, sequence i) corresponds to the sequence from position 8 to position 36 in a sequence selected from the group consisting of SEQ ID NO:1-7 presented in U.S. Pat. No. 10,934,335, which is incorporated by reference in its entirety. In one embodiment, sequence i) corresponds to the sequence from position 8 to position 36 in a sequence selected from the group consisting of SEQ ID NO: 1-4 presented in U.S. Pat. No. 10,934,335, which is incorporated by reference in its entirety. In one embodiment, sequence i) corresponds to the sequence from position 8 to position 36 in SEQ ID NO:1 presented in U.S. Pat. No. 10,934,335, which is incorporated by reference in its entirety.

In some embodiments of the present disclosure, the BM as defined above “forms part of” a three-helix bundle protein domain. This is understood to mean that the sequence of the BM is “inserted” into or “grafted” onto the sequence of the original three-helix bundle domain, such that the BM replaces a similar structural motif in the original domain. For example, without wishing to be bound by theory, the BM is thought to constitute two of the three helices of a three-helix bundle, and can therefore replace such a two-helix motif within any three-helix bundle. As the skilled person will realize, the replacement of two helices of the three-helix bundle domain by the two BM helices has to be performed so as not to affect the basic structure of the polypeptide. That is, the overall folding of the Ca backbone of the polypeptide according to this embodiment of the invention is substantially the same as that of the three-helix bundle protein domain of which it forms a part, e.g. having the same elements of secondary structure in the same order etc. Thus, a BM according to the disclosure “forms part” of a three-helix bundle domain if the polypeptide according to this embodiment of the aspect has the same fold as the original domain, implying that the basic structural properties are shared, those properties e.g. resulting in similar CD spectra. The skilled person is aware of other parameters that are relevant.

In particular embodiments, the IL-17A binding motif (BM) thus forms part of a three-helix bundle protein domain. For example, the BM may essentially constitute two alpha helices with an interconnecting loop, within said three-helix bundle protein domain. In particular embodiments, said three-helix bundle protein domain is selected from domains of bacterial receptor proteins. Non-limiting examples of such domains are the five different three-helical domains of Protein A from Staphylococcus aureus, such as domain B, and derivatives thereof. In some embodiments, the three-helical bundle protein domain is a variant of protein Z, which is derived from domain B of staphylococcal Protein A.

In some embodiments where the IL-17A binding polypeptide as disclosed herein forms part of a three-helix bundle protein domain, the IL-17A binding polypeptide may comprise an amino acid sequence binding module (BMod) selected from:
iii) K-[BM]-DPSQS XaXbLLXc EAKKL XdXeXIQ (SEQ ID NO:1296), as presented in U.S. Pat. No. 10,934,335, which is incorporated by reference in its entirety,
   wherein
   [BM] is an IL-17A binding motif as defined herein, provided that X₂₉ is D;
   Xₐ is selected from A and S;
   X_{b} is selected from N and E;
   X_{c} is selected from A, S and C;
   Xₐ is selected from E, N and S;
   Xₑ is selected from D, E and S;
   X_{f} is selected from A and S; and
iv) an amino acid sequence which has at least 85% identity to a sequence defined by iii).

It may be beneficial in some embodiments that said polypeptides exhibit high structural stability, such as resistance to chemical modifications, changes in physical conditions and proteolysis, during production or storage, as well as in vivo. Thus, in other embodiments where the IL-17A binding polypeptide as disclosed herein forms part of a three-helix bundle protein domain, the IL-17A binding polypeptide may comprise an amino acid sequence binding module (BMod) selected from:
v) K-[BM]-QPEQS XₐX_{b}LLX_{c}EAKKL X_{d}XₑX_{f}Q (SEQ ID NO:1297), as presented in U.S. Pat. No. 10,934,335, which is incorporated by reference in its entirety,
   wherein
   [BM] is an IL-17A binding motif as defined herein, provided that X₂₉ is R;
   Xₐ is selected from A and S;
   X_{b} is selected from N and E;
   X_{c} is selected from A, S and C;
   Xₐ is selected from E, N and S;
   Xₑ is selected from D, E and S;
   X_{f} is selected from A and S; and
   vi) an amino acid sequence which has at least 85% identity to a sequence defined by v).

As discussed above, polypeptides comprising minor changes as compared to the above amino acid sequences, which changes do not largely affect the tertiary structure or function of the polypeptide, are also within the scope of the present disclosure. Thus, in some embodiments, sequence iv and vi) have at least at least 87%, such as at least 89%, such as at least 91%, such as at least 93%, such as at least 95%, such as at least 97% identity to a sequence defined by iii) or v), respectively.

In one embodiment, Xₐ in sequence iii) or v) is A.

In one embodiment, Xₐ in sequence iii) or v) is S.

In one embodiment, X_{b} in sequence iii) or v) is N.

In one embodiment, X_{b} in sequence iii) or v) is E.

In one embodiment, X_{c} in sequence iii) or v) is A.

In one embodiment, X_{c} in sequence iii) or v) is S.

In one embodiment, X_{c} in sequence iii) or v) is C.

In one embodiment, X_{d} in sequence iii) or v) is E.

In one embodiment, X_{d} in sequence iii) or v) is N.

In one embodiment, X_{d} in sequence iii) or v) is S.

In one embodiment, Xₑ in sequence iii) or v) is D.

In one embodiment, Xₑ in sequence iii) or v) is E.

In one embodiment, Xₑ in sequence iii) or v) is S.

In one embodiment, X_{d}Xₑ in sequence iii) or v) is selected from EE, ES, SD, SE and SS.

In one embodiment, X_{d}Xₑ in sequence iii) or v) is ES.

In one embodiment, X_{d}Xₑ in sequence iii) or v) is SE.

In one embodiment, X_{d}Xₑ in sequence iii) or v) is SD.

In one embodiment, X_{f} in sequence iii) or v) is A.

In one embodiment, X_{f} in sequence iii) or v) is S.

In one embodiment, in sequence iii) or v), Xₐ is A; X_{b} is N; X_{c} is A and X_{f} is A.

In one embodiment, in sequence iii) or v), Xₐ is S, X_{b} is E; X_{c} is A and X_{f} is A.

In one embodiment, in sequence iii) or v), Xₐ is A; X_{b} is N; Xₑ is C and X_{f} is A.

In one embodiment, in sequence iii) or v), Xₐ is S, X_{b} is E; X_{c} is S and X_{f} is S.

In one embodiment, in sequence iii) or v), Xₐ is S, X_{b} is E; Xₑ is S and X_{f} is A.

In one embodiment, in sequence iii) or v), Xₐ is S, X_{b} is E; X_{c} is A and X_{f} is S.

In one embodiment, in sequence iii) or v), Xₐ is S, X_{b} is E; X_{c} is C and X_{f} is S.

In one embodiment, in sequence iii) or v), Xₐ is A; X_{b} is N; Xₑ is A; X_{d}Xₑ is ND and X_{f} is A.

In one embodiment, in sequence iii) or v), Xₐ is S, X_{b} is E; X_{c} is A; X_{d}Xₑ is ND and X_{f} is A.

In one embodiment, in sequence iii) or v), Xₐ is A; X_{b} is N; X_{c} is C; X_{d}Xₑ is ND and X_{f} is A.

In one embodiment, in sequence iii) or v), Xₐ is S, X_{b} is E; X_{c} is S, X_{d}Xₑ is ND and X_{f} is S.

In one embodiment, in sequence iii) or v), Xₐ is S, X_{b} is E; X_{c} is S, X_{d}Xₑ is ND and X_{f} is A.

In one embodiment, in sequence iii) or v), Xₐ is S, X_{b} is E; X_{c} is C; X_{d}Xₑ is ND and X_{f} is S.

In one embodiment, in sequence iii) or v), Xₐ is A; X_{b} is N; X_{c} is A; X_{d}Xₑ is SE and X_{f} is A.

In one embodiment, in sequence iii) or v), Xₐ is S, X_{b} is E; X_{c} is A; X_{d}Xₑ is SE and X_{f} is A.

In one embodiment, in sequence iii) or v), Xₐ is A; X_{b} is N; X_{c} is C; X_{d}Xₑ is SE and X_{f} is A.

In one embodiment, in sequence iii) or v), Xₐ is S, X_{b} is E; X_{c} is S, X_{d}Xₑ is SE and X_{f} is S.

In one embodiment, in sequence iii) or v), Xₐ is S, X_{b} is E; Xₑ is A; X_{d}Xₑ is SE and X_{f} is S.

In one embodiment, in sequence iii) or v), Xₐ is S, X_{b} is E; Xₑ is C; X_{d}Xₑ is SE and X_{f} is S.

In one embodiment, in sequence iii) or v), Xₐ is A; X_{b} is N; X_{c} is A; X_{d}Xₑ is ES and X_{f} is A.

In one embodiment, in sequence iii) or v), Xₐ is S, X_{b} is E; X_{c} is A; X_{d}Xₑ is ES and X_{f} is A.

In one embodiment, in sequence iii) or v), Xₐ is A; X_{b} is N; X_{c} is C; X_{d}Xₑ is ES and X_{f} is A.

In one embodiment, in sequence iii) or v), Xₐ is S, X_{b} is E; X_{c} is S, X_{d}Xₑ is ES and X_{f} is S.

In one embodiment, in sequence iii) or v), Xₐ is S, X_{b} is E; X_{c} is C; X_{d}Xₑ is ES and X is S.

In one embodiment, in sequence iii) or v), Xₐ is A; X_{b} is N; X_{c} is A; X_{d}Xₑ is SD and X_{f} is A.

In one embodiment, in sequence iii) or v), Xₐ is S, X_{b} is E; X_{c} is A; X_{d}Xₑ is SD and X_{f} is A.

In one embodiment, in sequence iii) or v), Xₐ is A; X_{b} is N: X_{c} is C; X_{d}Xₑ is SD and X_{f} is A.

In one embodiment, in sequence iii) or v), Xₐ is S, X_{b} is E; X_{c} is S, X_{d}Xₑ is SD and X_{f} is S.

In one embodiment, in sequence iii) or v), Xₐ is S, X_{b} is E; X_{c} is A; X_{d}Xₑ is SD and X_{f} is S.

In one embodiment, in sequence iii) or v), Xₐ is S, X_{b} is E; X_{c} is C; X_{d}Xₑ is SD and X_{f} is S.

In yet a further embodiment, sequence iii) corresponds to the sequence from position 7 to position 55 in a sequence selected from the group consisting of SEQ ID NO:1-1216 presented in FIG. 1 of U.S. Pat. No. 10,934,335, which is incorporated by reference in its entirety. In one embodiment, sequence iii) corresponds to the sequence from position 7 to position 55 in a sequence selected from the group consisting of SEQ ID NO:1-66, 1200, 1206 and 1214, presented in FIG. 1 of U.S. Pat. No. 10,934,335, which is incorporated by reference in its entirety. In one embodiment, sequence iii) corresponds to the sequence from position 7 to position 55 in a sequence selected from the group consisting of SEQ ID NO:1-66, presented in U.S. Pat. No. 10,934,335, which is incorporated by reference in its entirety. In one embodiment, sequence iii) corresponds to the sequence from position 7 to position 55 in a sequence selected from the group consisting of SEQ ID NO:1-35, presented in U.S. Pat. No. 10,934,335, which is incorporated by reference in its entirety. In another embodiment, sequence iii) corresponds to the sequence from position 7 to position 55 in a sequence selected from the group consisting of SEQ ID NO: 1-27 presented in U.S. Pat. No. 10,934,335, which is incorporated by reference in its entirety. In one embodiment, sequence iii) corresponds to the sequence from position 7 to position 55 in a sequence selected from the group consisting of SEQ ID NO:1-10 presented in U.S. Pat. No. 10,934,335, which is incorporated by reference in its entirety. In yet another embodiment, sequence iii) corresponds to the sequence from position 7 to position 55 in a sequence selected from the group consisting of SEQ ID NO:1-7 presented in U.S. Pat. No. 10,934,335, which is incorporated by reference in its entirety. In one embodiment, sequence iii) corresponds to the sequence from position 7 to position 55 in a sequence selected from the group consisting of SEQ ID NO:1-4 and in another embodiment, sequence iii) corresponds to the sequence from position 7 to position 55 in SEQ ID NO: 1 presented in U.S. Pat. No. 10,934,335, which is incorporated by reference in its entirety.

Also, in a further embodiment, there is provided an IL-17A binding polypeptide, which comprises an amino acid sequence selected from:
vii) YA-[BMod]-AP (SEQ ID NO:1298), presented in U.S. Pat. No. 10,934,335, which is incorporated by reference in its entirety wherein [BMod] is an IL-17A binding module as defined above; and
viii) an amino acid sequence which has at least 86% identity to a sequence defined by vii).

In an alternative further embodiment, there is provided an IL-17A binding polypeptide, which comprises an amino acid sequence selected from:
ix) FA-[BMod]-AP (SEQ ID NO:1299), presented in U.S. Pat. No. 10,934,335, which is incorporated by reference in its entirety,
wherein [BMod] is an IL-17A binding module as defined above; and
x) an amino acid sequence which has at least 86% identity to a sequence defined by ix).

Alternatively, there is provided an IL-17A binding polypeptide, which comprises an amino acid sequence selected from:
xi) FN-[BMod]-AP (SEQ ID NO:1300) presented in U.S. Pat. No. 10,934,335, which is incorporated by reference in its entirety,
wherein [BMod] is an IL-17A binding module as defined above; and
xii) an amino acid sequence which has at least 86% identity to a sequence defined by xi).

As discussed above, polypeptides comprising minor changes as compared to the above amino acid sequences without largely affecting the tertiary structure and the function thereof also fall within the scope of the present disclosure. Thus, in some embodiments, the IL-17A binding polypeptides as defined above may for example have a sequence which is at least 88%, such as at least 90%, such as at least 92%, such as at least 94%, such as at least 96%, such as at least 98% identical to a sequence defined by vii), ix) or xi).

In some embodiments, the IL-17A binding motif may form part of a polypeptide comprising an amino acid sequence selected from:

| SEQ | |
|---|---|
| ID NO: | SEQUENCE |
| 10 | ADNNFNK-[BM]-DPSQSANLLSEAKKLNESQAPK; |
| 11 | ADNKFNK-[BM]-DPSQSANLLAEAKKLNDAQAPK; |
| 13 | ADNKFNK-[BM]-DPSVSKEILAEAKKLNDAQAPK; |
| 15 | ADAQQNNFNK-[BM]-DPSQSTNVLGEAKKLNESQAPK; |
| 16 | AQHDE-[BM]-DPSQSANVLGEAQKLNDSQAPK; |
| 17 | VDNKFNK-[BM]-DPSQSANLLAEAKKLNDAQAPK; |
| 18 | AEAKYAK-[BM]-DPSESSELLSEAKKLNKSQAPK; |
| 19 | VDAKYAK-[BM]-DPSQSSELLAEAKKLNDAQAPK; |
| 20 | VDAKYAK-[BM]-DPSQSSELLAEAKKLNDSQAPK; |
| 21 | AEAKYAK-[BM]-DPSQSSELLSEAKKLNDSQAPK; |
| 22 | AEAKYAK-[BM]-DPSQSSELLSEAKKLNDSQAP; |
| 23 | AEAKYAK-[BM]-DPSQSSELLSEAKKLNDAQAPK; |
| 24 | AEAKYAK-[BM]-DPSQSSELLSEAKKLNDAQAP; |
| 25 | AEAKFAK-[BM]-DPSQSSELLSEAKKLNDSQAPK; |
| 26 | AEAKFAK-[BM]-DPSQSSELLSEAKKLNDSQAP; |
| 27 | AEAKYAK-[BM]-DPSQSSELLAEAKKLNDAQAPK; |
| 28 | AEAKYAK-[BM]-DPSQSSELLSEAKKLSESQAPK; |
| 29 | AEAKYAK-[BM]-DPSQSSELLSEAKKLSESQAP; |
| 30 | AEAKFAK-[BM]-DPSQSSELLSEAKKLSESQAPK; |
| 31 | AEAKFAK-[BM]-DPSQSSELLSEAKKLSESQAP; |
| 32 | AEAKYAK-[BM]-DPSQSSELLAEAKKLSEAQAPK; |
| 33 | AEAKYAK-[BM]-QPEQSSELLSEAKKLSESQAPK; |
| 34 | AEAKYAK-[BM]-DPSQSSELLSEAKKLESSQAPK; |
| 35 | AEAKYAK-[BM]-DPSQSSELLSEAKKLESSQAP; |
| 36 | AEAKYAK-[BM]-DPSQSSELLAEAKKLESAQAPK; |
| 37 | AEAKYAK-[BM]-QPEQSSELLSEAKKLESSQAPK; |
| 38 | AEAKYAK-[BM]-DPSQSSELLSEAKKLSDSQAPK; |
| 39 | AEAKYAK-[BM]-DPSQSSELLSEAKKLSDSQAP; |
| 40 | AEAKYAK-[BM]-DPSQSSELLAEAKKLSDSQAPK; |
| 41 | AEAKYAK-[BM]-DPSQSSELLAEAKKLSDAQAPK; |
| 42 | AEAKYAK-[BM]-QPEQSSELLSEAKKLSDSQAPK; |
| 43 | VDAKYAK-[BM]-DPSQSSELLSEAKKLNDSQAPK; |
| 44 | VDAKYAK-[BM]-DPSQSSELLSEAKKLSESQAPK; |
| 45 | VDAKYAK-[BM]-DPSQSSELLAEAKKLSEAQAPK; |
| 46 | VDAKYAK-[BM]-QPEQSSELLSEAKKLSESQAPK; |
| 47 | VDAKYAK-[BM]-DPSQSSELLSEAKKLESSQAPK; |
| 48 | VDAKYAK-[BM]-DPSQSSELLAEAKKLESAQAPK; |
| 49 | VDAKYAK-[BM]-QPEQSSELLSEAKKLESSQAPK; |
| 50 | VDAKYAK-[BM]-DPSQSSELLSEAKKLSDSQAPK; |
| 51 | VDAKYAK-[BM]-DPSQSSELLAEAKKLSDSQAPK; |
| 52 | VDAKYAK-[BM]-DPSQSSELLAEAKKLSDAQAPK; |
| 53 | VDAKYAK-[BM]-QPEQSSELLSEAKKLSDSQAPK; |
| 54 | VDAKYAK-[BM]-DPSQSSELLAEAKKLNKAQAPK; |
| 55 | AEAKYAK-[BM]-DPSQSSELLAEAKKLNKAQAPK; and |
| 56 | ADAKYAK-[BM]-DPSQSSELLSEAKKLNDSQAPK; |

wherein [BM] is an IL-17A binding motif as defined above.

In one embodiment, the IL-17A binding polypeptide comprises an amino acid sequence selected from:
xiii) VDAKYAK-[BM]-DPSQSSELLSEAKKLNDSQAPK (SEQ ID NO:1281), presented in U.S. Pat. No. 10,934,335, which is incorporated by reference in its entirety wherein [BM] is an IL-17A binding motif as defined above; and
xiv) an amino acid sequence which has at least 86% identity to the sequence defined in xiii).

Again, polypeptides comprising minor changes as compared to the above amino acid sequences without largely affecting the tertiary structure and the function thereof are also within the scope of the present disclosure. Thus, in some embodiments, the IL-17A binding polypeptides as defined above may for example have a sequence which is at least 87%, such as at least 89%, such as at least 91%, such as at least 93%, such as at least 94%, such as at least 96%, such as at least 98% identical to the sequence defined by xiii).

Sequence xiii) in such a polypeptide may be selected from the group consisting of SEQ ID NO: 1-1216, presented in U.S. Pat. No. 10,934,335, which is incorporated by reference in its entirety. In one embodiment, sequence xiii) is selected from the group consisting of SEQ ID NO:1-66, 1200, 1206 and 1214, presented in U.S. Pat. No. 10,934,335, which is incorporated by reference in its entirety. In one embodiment, sequence xiii) is selected from the group consisting of SEQ ID NO: 1-66, presented in U.S. Pat. No. 10,934,335, which is incorporated by reference in its entirety. In one embodiment, sequence xiii) is selected from the group consisting of SEQ ID NO: 1-35, presented in U.S. Pat. No. 10,934,335, which is incorporated by reference in its entirety. In another embodiment, sequence xiii) is selected from the group consisting of SEQ ID NO: 1-27, presented in U.S. Pat. No. 10,934,335, which is incorporated by reference in its entirety. In one embodiment, sequence xiii) is selected from the group consisting of SEQ ID NO:1-10, presented in U.S. Pat. No. 10,934,335, which is incorporated by reference in its entirety. In one embodiment, sequence xiii) is selected from SEQ ID NO:1-7, presented in U.S. Pat. No. 10,934,335, which is incorporated by reference in its entirety. In one embodiment, sequence xiii) is selected from the group consisting of SEQ ID NO: 1-4, presented in U.S. Pat. No. 10,934,335, which is incorporated by reference in its entirety. In one embodiment, sequence xiii) is SEQ ID NO: 1, presented in U.S. Pat. No. 10,934,335, which is incorporated by reference in its entirety.

In one embodiment, the IL-17A binding polypeptide comprises an amino acid sequence selected from:
xv) AEAKYAK-[BM]-DPSQSSELLSEAKKLNDSQAPK (SEQ ID NO:1259), presented in U.S. Pat. No. 10,934,335, which is incorporated by reference in its entirety, wherein [BM] is an IL-17A binding motif as defined above; and
xvi) an amino acid sequence which has at least 86% identity to the sequence defined in xv).

Again, polypeptides comprising minor changes as compared to the above amino acid sequences without largely affecting the tertiary structure and the function thereof are also within the scope of the present disclosure. Thus, in some embodiments, the IL-17A binding polypeptides as defined above may for example have a sequence which is at least 87%, such as at least 89%, such as at least 91%, such as at least 93%, such as at least 94%, such as at least 96%, such as at least 98% identical to the sequence defined by xv).

Sequence xv) in such a polypeptide may be selected from the group consisting of SEQ ID NO: 1217-1222, presented in U.S. Pat. No. 10,934,335, which is incorporated by reference in its entirety. In one embodiment, sequence xv) is selected from the group consisting of SEQ ID NO: 1218-1222, presented in U.S. Pat. No. 10,934,335, which is incorporated by reference in its entirety. In one embodiment, sequence xv) is selected from the group consisting of SEQ ID NO: 1219-1222, presented in U.S. Pat. No. 10,934,335, which is incorporated by reference in its entirety. In another embodiment, sequence xv) is selected from the group consisting of SEQ ID NO: 1219 and SEQ ID NO:1222, presented in U.S. Pat. No. 10,934,335, which is incorporated by reference in its entirety. In one embodiment, sequence xv) is SEQ ID NO:1219, presented in U.S. Pat. No. 10,934,335, which is incorporated by reference in its entirety.

The small size and robustness of the IL-17A binding domains of the present disclosure confer several advantages over conventional monoclonal antibody-based therapies. Such advantages include the possibility of subcutaneous (s.c.) administration at higher doses than antibodies, alternative routes of administration, flexibility in formatting for superior potency and absence of Fc-mediated side effects. The small size combined with potential for very high solubility (>100 mg/ml) and stability allows for extreme molar amounts of drug in a small volume for s.c. injections. For systemic administration, this suggests outpatient “home use” treatment using convenient small prefilled syringes or auto-injectors, with low volume and well tolerated administration of doses. In addition, the capacity for high molar concentrations in drug preparations in combination with the ability to retain functional stability in diverse formulations opens up for topical (skin, eye, lung) administration routes. Psoriasis, asthma, uveitis and dry eye syndrome are examples of indications where alternative administration routes could be especially relevant in IL-17A mediated disease.

The IL-17A binding motif for the bispecific fusion protein of the invention are disclosed in U.S. Pat. No. 10,934,335 and U.S. Patent Publication No. 2021/0253659, which are hereby incorporated in their entirety by reference herein.

### Albumin binding motif

In another embodiment of the invention, the albumin binding motif consists of an amino acid sequence selected from (SEQ ID. NO: 2)
xvii) LAX₃AKX₆X-ANX₁₀ELDX₁₄YGVSDFYKRLIX₂₆KAKTVEGVEALKX₃₉X₄₀IL X₄₃X₄LP;
   wherein independently of each other
   X₃ is selected from E, S, Q and C;
   X₆ is selected from E, S and C,
   X₇is selected from A and S;
   X₁₀ is selected from A, S and R;
   X₁₄ is selected from A, S, C and K;
   X₂₆ is selected from D and E;
   X₃₉ is selected from D and E;
   X₄₀ is selected from A and E;
   X₄₃ is selected from A and K.
   X₄₄ is selected from A, S and E;
   L in position 45 is present or absent; and
   P in position 46 is present or absent; and
xviii) an amino acid sequence which has at least 95% identity to the sequence defined in xvii).

The above defined class of sequence related polypeptides having a binding affinity for albumin is derived from a common parent polypeptide sequence, which folds into a three alpha helix bundle domain. More specifically, the polypeptides as described above are derived from a model building based on a structure of a complex between serum albumin and the albumin binding domain G148-GA3 (Lejon et al, J Biol Chem 279:42924-8, 2004), as well as analyses of binding and structural properties of a number of mutational variants of the common parent polypeptide sequence. The above defined amino acid sequence xvii) comprises amino acid substitutions as compared to the parent polypeptide sequence that result in a class of polypeptides which are expected to fold into an almost identical three helix bundle domain. While the parent polypeptide sequence already comprises a binding surface for interaction with albumin, that binding surface is modified by some of the substitutions according to the above definition. The substitutions according to the above definition provide an improved albumin binding ability as compared to the parent polypeptide sequence.

In certain embodiments, the albumin binding polypeptides exhibit a set of characteristics, which, for example, make them suitable for use as fusion or conjugate partners for therapeutic molecules for human administration. The albumin binding polypeptides according to the present disclosure demonstrate, for example in comparison with related albumin binding polypeptides such as the albumin binding domain G148-GA3 and the albumin binding polypeptides disclosed in WO 09/016,043, at least five of the following six characteristics:
The polypeptides display a different surface compared to, for example, G148-GA3 and other bacterially derived albumin binding domains. The difference may decrease or eliminate any risk for antibody reactions in a subject, such as a human, which has been previously exposed to such bacterial proteins.
The polypeptides comprise fewer potential T-cell epitopes than, for example, G148-GA3 and other related, but different, mutational variants of the common parent polypeptide sequence, and hence exhibit low immunogenicity when administered to a subject, such as a human.
The polypeptides display a lower reactivity with circulating antibodies when administered to a subject, such as a human. Thus, by amino acid substitutions in the surface of the polypeptides exposed to circulating antibodies, i.e. in the polypeptide surface not involved in the binding interaction with albumin, antibody cross-reactivity is reduced as compared to, for example, antibody cross-reactivity caused by G148-GA3 as measured in a test set of human sera.
The polypeptides have a high albumin binding ability, both in terms of a higher binding affinity, as defined by a Kp value, and in terms of a slower off-rate, as defined by a Koff value, than, for example, known naturally occurring albumin binding polypeptides, such as the albumin binding domains derived from bacterial proteins.
The polypeptides comprise fewer amino acid residues that are associated with stability problems of polypeptides than, for example, known naturally occurring albumin binding polypeptides, such as the albumin binding domains derived from bacterial proteins. Thus, the polypeptides comprise, for example, no oxidation-prone methionines or tryptophanes and only one asparagine.
The polypeptides have a higher structural stability, as defined by a melting point of above 55° C., than previous albumin binding polypeptides, such as those disclosed in WO 09/016,043.

In another embodiment, the albumin binding motif displays all six of the above listed characteristics. In another embodiment, the albumin binding motif, when bound to albumin, a more hydrophilic profile than, for example, previous albumin binding polypeptides, such as those disclosed in WO 09/016,043. The surface of the albumin binding polypeptide which is exposed to the surroundings when the polypeptide interacts with albumin comprises fewer amino acid residues that confer surface hydrophobicity.

As the skilled person will realize, the function of any polypeptide, such as the albumin binding capacity of the polypeptides, is dependent on the tertiary structure of the polypeptide. It is however possible to make changes to the sequence of amino acids in an a-helical polypeptide without affecting the structure thereof (Taverna and Goldstein, J Mol Biol 315 (3):479-84, 2002; He et al, Proc Natl Acad Sci USA 105(38):14412-17, 2008). Thus, a person of ordinary skill in the art would recognize that the modified variants of i), which are such that the resulting sequence is at least 95% identical to a sequence belonging to the class defined by i), are also encompassed by the current invention. For example, it is possible that an amino acid residue belonging to a certain functional grouping of amino acid residues (e.g. hydrophobic, hydrophilic, polar etc) could be exchanged for another amino acid residue from the same functional group.

The term “% identical” or “% identity”, as used in the specification and claims, is calculated as follows. The query sequence is aligned to the target sequence using the CLUSTAL W algorithm (Thompson, J. D., Higgins, D. G. and Gibson, T. J., Nucleic Acids Research, 22:4673-4680 (1994)). A comparison is made over the window corresponding to the shortest of the aligned sequences. The shortest of the aligned sequences may in some instances be the target sequence, such as the albumin binding polypeptide disclosed herein. In other instances, the query sequence may constitute the shortest of the aligned sequences. The query sequence may for example consist of at least 10 amino acid residues, such as at least 20 amino acid residues, such as at least 30 amino acid residues, such as at least 40 amino acid residues, for example 45 amino acid residues. The amino acid residues at each position are compared, and the percentage of positions in the query sequence that have identical correspondences in the target sequence is reported as % identity.

In one embodiment of the albumin binding motif, described in xvii), X₆ is E.

In another embodiment of the albumin binding motif, described in xvii), X₃ is S.

In another embodiment of the albumin binding motif, described in xvii), X₃ is E.

In another embodiment of the albumin binding motif, described in xvii), X₇is A.

In another embodiment of the albumin binding motif, described in xvii), X₁₄is S.

In another embodiment of the albumin binding motif, described in xvii), X₁₄ is C.

In another embodiment of the albumin binding motif, described in xvii), X₁₀ is A.

In another embodiment of the albumin binding motif, described in xvii), X₁₀ is S.

In another embodiment of the albumin binding motif, described in xvii), X₂₆ is D.

In another embodiment of the albumin binding motif, described in xvii), X₂₆ is E.

In another embodiment of the albumin binding motif, described in xvii), X₃₉ is D.

In another embodiment of the albumin binding motif, described in xvii), X₃₉ is E.

In another embodiment of the albumin binding motif, described in xvii), X₄₀ is A.

In another embodiment of the albumin binding motif, described in xvii), X₄₃ is A.

In another embodiment of the albumin binding motif, described in xvii), X₄₄ is A.

In another embodiment of the albumin binding motif, described in xvii), X₄₄ is S.

In another embodiment of the albumin binding motif, the L residue in position 45 is present.

In another embodiment of the albumin binding motif, described in xvii), the P residue in position 46 is present.

In another embodiment of the albumin binding motif, described in xvii), the P residue in position 46 is absent.

In another embodiment, the albumin binding polypeptide, described in xvii), is subject to the proviso that X₇is neither L, E nor D.

The albumin binding polypeptide may be prepared for conjugation with a suitable conjugation partner by the replacement of surface exposed amino acid residues with, for example, either a cysteine or a lysine. These replacements may be introduced into the N-terminal helix, i.e. helix one, of the polypeptide, which is the helix situated furthest away from the serum albumin when the albumin binding polypeptide is bound to serum albumin. Thus, a lysine residue in position X₁₄ of the sequence defined in i) may be used to enable site-directed conjugation. This may furthermore be advantageous when the molecule is made by chemical peptide synthesis, since orthogonal protection of the epsilon-amino group of said lysine may be utilized. Furthermore, a cysteine residue may be introduced into the amino acid sequence to enable site-directed conjugation. For example, a cysteine residue may be introduced into any one of the positions X₃, X₆ and/or X₁₄ in accordance with the above definition.

Coupling of a conjugation partner to the epsilon-amine of a lysine or the thiol group of a cysteine represents two chemically different alternatives to obtain site-directed conjugation using an amino acid residue within the amino acid sequence xvii). As the skilled person understands, other chemical alternatives for preparing an amino acid sequence for conjugation exist, and are as such also within the scope of the present disclosure. One example of such a chemistry is the click-like chemistry enabled by the introduction of a tyrosine as presented by Ban et al (J Am Chem Soc 132:1523-5, 2009).

In one embodiment, the albumin binding polypeptide comprises one or more additional amino acid residues positioned at the N- and/or the C-terminal of the sequence defined in xvii). These additional amino acid residues may play a role in enhancing the binding of albumin by the polypeptide, and improving the conformational stability of the folded albumin binding domain, but may equally well serve other purposes, related for example to one or more of production, purification, stabilization in vivo or in vitro, coupling, labeling or detection of the polypeptide, as well as any combination thereof. Such additional amino acid residues may comprise one or more amino acid residue(s) added for purposes of chemical coupling, e.g. to a chromatographic resin to obtain an affinity matrix or to a chelating moiety for complexing with a radiometal.

The amino acids directly preceding or following the alpha helix at the N- or C-terminus of the amino acid sequence xvii) may thus in one embodiment affect the conformational stability. One example of an amino acid residue which may contribute to improved conformational stability is a serine residue positioned at the N-terminal of the amino acid sequence i) as defined above. The N-terminal serine residue may in some cases form a canonical S-X-X-E capping box, by involving hydrogen bonding between the gamma oxygen of the serine side chain and the polypeptide backbone NH of the glutamic acid residue. This N-terminal capping may contribute to stabilization of the first alpha helix of the three helix domain constituting the albumin binding polypeptide.

Thus, in one embodiment, the additional amino acids comprise at least one serine residue at the N-terminal of the polypeptide. The amino acid sequence is in other words preceded by one or more serine residue(s). In another embodiment of the albumin binding polypeptide, the additional amino acids comprise a glycine residue at the N-terminal of the polypeptide. It is understood that the amino acid sequence xvii) may be preceded by one, two, three, four or any suitable number of amino acid residues. Thus, the amino acid sequence may be preceded by a single serine residue, a single glycine residue or a combination of the two, such as a glycine-serine (GS) combination or a glycine-serine-serine (GSS) combination. Examples of albumin binding polypeptides comprising additional amino residues at the N-terminal are set out in SEQ ID NO: 145-163, such as in SEQ ID NO: 145-148 and SEQ ID NO: 162-163, as presented in U.S. Pat. No. 9,211,344, which is incorporated by reference in its entirety. In yet another embodiment, the additional amino acid residues comprise a glutamic acid at the N-terminal of the polypeptide as defined by the sequence i).

Similarly, C-terminal capping may be exploited to improve stability of the third alpha helix of the three helix domain constituting the albumin binding polypeptide. A proline residue, when present at the C-terminal of the amino acid sequence defined in i), may at least partly function as a capping residue. In such a case, a lysine residue following the proline residue at the C-terminal may contribute to further stabilization of the third helix of the albumin binding polypeptide, by hydrogen bonding between the epsilon amino group of the lysine residue and the carbonyl groups of the amino acids located two and three residues before the lysine in the polypeptide backbone, e.g., when both L45 and P46 are present, the carbonyl groups of the leucine and alanine residues of the amino acid sequence defined in xvii). Thus, in one embodiment, the additional amino acids comprise a lysine residue at the C-terminal of the polypeptide.

As discussed above, the additional amino acids may be related to the production of the albumin binding polypeptide. In particular, when an albumin binding polypeptide according to an embodiment in which P46 is present is produced by chemical peptide synthesis, one or more optional amino acid residues following the C-terminal proline may provide advantages. Such additional amino acid residues may for example prevent formation of undesired substances, such as diketopiperazine at the dipeptide stage of the synthesis. One example of such an amino acid residue is glycine. Thus, in one embodiment, the additional amino acids comprise a glycine residue at the C-terminal of the polypeptide, directly following the proline residue or following an additional lysine and/or glycine residue as accounted for above. Alternatively, polypeptide production may benefit from amidation of the C-terminal proline residue of the amino acid sequence i), when present. In this case, the C-terminal proline comprises an additional amine group at the carboxyl carbon. In one embodiment of the polypeptides described herein, particularly those ending at its C-terminus with proline or other amino acid known to racemize during peptide synthesis, the above-mentioned addition of a glycine to the C-terminus or amidation of the proline, when present, can also counter potential problems with racemization of the C-terminal amino acid residue. If the polypeptide, amidated in this way, is intended to be produced by recombinant means, rather than by chemical synthesis, amidation of the C-terminal amino acid can be performed by several methods known in the art, e.g. through the use of amidating PAM enzyme.

The albumin binding motifs for the bispecific fusion protein of the invention are disclosed in U.S. Pat. Nos. 9,211,344, 10,329,331, 8,937,153, and 10,118,949, which are hereby incorporated in their entirety by reference herein.

### Pharmaceutical Compositions

Another aspect of the invention provides for a pharmaceutical composition comprising a bispecific fusion protein, preferably izokibep. In another aspect of the invention, the pharmaceutical composition comprises additional excipients. The pharmaceutical composition may be in a form suitable for oral use, for example, as tablets, troches, lozenges, fast-melts, aqueous or oily suspensions, dispersible powders or granules, emulsions, hard or soft capsules, syrups, or elixirs. Compositions intended for oral use may be prepared according to any method known in the art for the manufacture of pharmaceutical compositions and such compositions may contain one or more agents selected from sweetening agents, flavoring agents, coloring agents, and preserving agents, in order to provide pharmaceutically elegant and palatable preparations. Depending on the specific conditions being treated, such agents may be formulated into liquid or solid dosage forms and administered systemically or locally. The agents may be delivered, for example, in a timed-or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

The composition may be provided according to a dosing regimen. A dosing regimen may include one or more of a dosage, a dosing frequency, and a duration.

### Dosing Regimen

In one aspect of the invention, the aforementioned bispecific fusion protein, wherein the bispecific fusion protein comprises an IL-17A binding motif and an albumin binding motif is administered to the patients suffering from several chronic inflammatory diseases such as psoriasis, rheumatoid arthritis (RA), ankylosing spondylitis (AS), systemic lupus erythematosus (SLE) and multiple sclerosis (MS) (Miossec and Kolls, 2012, Nat Rev Drug Discov 11:763-7).

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

In another aspect of the invention, the aforementioned bispecific fusion protein, wherein the bispecific fusion protein comprises an IL-17A binding motif and an albumin binding motif is administered to the patients suffering from ankylosing spondylitis (AS).

In another preferred aspect of the invention, a pharmaceutical composition comprising a therapeutically effective of amount of the bispecific fusion protein is administered to a patient suffering from AS. In another aspect, there is provided a composition comprising a bispecific fusion protein, as described herein, and at least one pharmaceutically acceptable excipient or carrier. In one embodiment, said composition further comprises at least one additional active agent, such as at least two additional active agents, such as at least three additional active agents. Non-limiting examples of additional active agents that may prove useful in such a composition are the therapeutically active polypeptides, immune response modifying agents and toxic compounds described herein.

In another aspect of the invention, a pharmaceutical composition comprising a therapeutically effective of amount of the bispecific fusion protein, preferably izokibep, is administered to a patient suffering from AS.

In another aspect of the invention, the pharmaceutical composition comprising a therapeutically effective of amount of the bispecific fusion protein, preferably izokibep, is administered by a subcutaneous injection.

In another aspect of the invention, the pharmaceutical composition comprises about 20 to about 400 mg izokibep.

In another aspect of the invention, the pharmaceutical composition comprises about 40 mg, about 80 mg, or about 160 mg izokibep.

In another aspect of the invention, the pharmaceutical composition comprising a therapeutically effective of amount of the bispecific fusion protein, preferably izokibep, is administered as a subcutaneous injection once weekly, twice weekly, or once every four weeks.

In another aspect of the invention, the pharmaceutical composition comprising a therapeutically effective of amount of the bispecific fusion protein, preferably izokibep, is administered as a subcutaneous injection is administered for at least 16 weeks.

In another aspect of the invention, the pharmaceutical composition comprising a therapeutically effective of amount of the bispecific fusion protein, preferably izokibep, is administered as a subcutaneous injection is administered for at least 52 weeks.

In another aspect of the invention, the pharmaceutical composition comprising about 40 mg izokibep is administered once a week to a patient suffering from AS.

In another aspect of the invention, the pharmaceutical composition comprising about 160 mg izokibep is administered once a week to a patient suffering from AS.

In another aspect of the invention, the administration of the pharmaceutical compositions comprising izokibep leads to a clinical response as assessed by Assessment of Spondyloarthritis International Society 40 (ASAS40) response after 16 weeks or 52 weeks in subjects with active AS. In a preferred embodiment, the patient suffering from AS achieves a ASA40 response after 16 weeks.

In another aspect of the invention, the patient is suffering from axial spondyloarthritis (AxSpA).

In another aspect of the invention, the patient is suffering from radiographic axial spondyloarthritis (r-AxSpA).

In another aspect of the invention, the patient is suffering from non-radiographic axial spondyloarthritis (nr-AxSpA).

In another aspect of the invention, the patient is administered the pharmaceutical compositions comprising izokibep suffering from AS had an inadequate response or intolerance to at least 2 Non-steroidal anti-inflammatory drugs (NSAIDs), or contraindication to NSAID therapy.

In another aspect of the invention, the patient is administered the pharmaceutical compositions comprising izokibep suffering from AS is TNFa inhibitor-naïve or may have received up to 2 prior TNFα inhibitor(s).

In another aspect of the invention, the patient is administered the pharmaceutical compositions comprising izokibep suffering from AS had an inadequate response to any previous therapies, including a therapy by a biomolecule, or any other Janus Kinase (JAK) inhibitors.

### Examples

The examples provided herein are representative for the dosing regimens disclosed in the invention. An exemplary clinical dosing in accordance with the present disclosure is provided below.

The study will include the following 3 periods:
1. Screening Period: Up to 35 days prior to baseline randomization.
2. Treatment Period 1: (Placebo-Controlled, Double-Blind Period): Day 0 to Week 16 Cohort 1: Eligible subjects will be randomized 1:1:1:1 to receive 1 of 4 treatments (Izokibep 160 mg once every two weeks, izokibep 40 mg once every two weeks, izokibep 160 mg once every four weeks, or placebo Q2W), and will remain on their allowable background medication.
   Cohort 2: Eligible subjects will be randomized 1:1:1 to receive 1 of 3 treatments (izokibep 160 mg once a week, izokibep 40 mg once a week, or placebo once a week), and will remain on their allowable background medication.
Treatment Period 1 ends at Week 16 after all trial assessments have been done and Treatment Period 2 starts at Week 16 with the IMP injection.
3. Treatment Period 2 (Open-label Extension Period): Week 16 to Week 52 Cohort 1: Subjects will receive izokibep 160 mg once every two weeks subcutaneous injection treatment in an open-label manner.; Cohort 2: Subjects will receive izokibep 160 mg once a week subcutaneous treatment in an open-label manner.

At week 24, subjects who could not achieve an ASAS20 response from baseline are defined as non-responders and will discontinue the study treatment.

### Inclusion Criteria

1. Male or female at least 18 years of age.
2. Subjects with active AS, determined by documented radiologic evidence (X-ray) fulfilling the Modified New York criteria for AS (1984) and at least one SpA feature, according to ASAS criteria.
3. Subjects have moderate to severe active disease
4. Subjects must have inadequate response or intolerance to at least 2 NSAIDs, or contraindication to NSAID therapy.
5. Subjects may be TNFa inhibitor-naïve or may have received up to 2 prior TNFa inhibitor(s).

### Exclusion Criteria

1. Subjects have active fibromyalgia or total spinal ankylosis (‘bamboo spine’), or any other inflammatory arthritis.
2. Subjects have used medications in the manner as detailed by the exclusion criteria as detailed in the study protocol.
3. Subjects have received technetium-99 conjugated with methylene diphosphonate other than for diagnostic purpose within 5 years prior to baseline.
4. Have received any live (includes attenuated) vaccination within the 12 weeks prior to the baseline.
5. Subjects have received any non-biological therapy for AS not listed as detailed in the study protocol within or outside a clinical study in the 3 months or within 5 half-lives prior to the Baseline Visit (whichever is longer).
6. Subject has an active infection or history of infections
7. Have evidence of or test positive for hepatitis B virus (HBV)
8. Have evidence of or test positive for hepatitis C virus (HCV).
9. Have a historically positive human immunodeficiency virus (HIV) test or test positive at screening for HIV.
10. Subjects have known tuberculosis (TB) infection, at high risk of acquiring TB infection, or current or history of nontuberculous mycobacterium (NTMB) infection, or LTB.
11. Have a history of a lymphoproliferative disorder including lymphoma or current signs and symptoms suggestive of lymphoproliferative disease.
12. Subjects have active Crohn's disease (CD) or active ulcerative colitis (UC).
13. Subjects have active uveitis within 6 weeks prior to baseline.
14. Subjects have laboratory abnormalities at Screening.

### Dosing Regimen

The dosing regimen in the various arms of clinical trials are provided below.

| Arm | Intervention/Treatment |
|---|---|
| Active Comparator: izokibep 160 mg once | Izokibep 160 mg once every two weeks, 160 |
| every two weeks; subcutaneous injection; | mg once every four weeks, 40 mg once every |
| Cohort 1 | two weeks; 160 mg once weekly, 40 mg once |
| | weekly; Duration: 52 weeks |
| Active Comparator: izokibep 40 mg once | Izokibep 160 mg once every two weeks, 160 |
| every two weeks; subcutaneous injection; | mg once every four weeks, 40 mg once every |
| Cohort 1 | two weeks; 160 mg once weekly, 40 mg once |
| | weekly; Duration: 52 weeks |
| Active Comparator: izokibep 160 mg once | Izokibep 160 mg once every two weeks, 160 |
| every four weeks; subcutaneous injection; | mg once every four weeks, 40 mg once every |
| Cohort 1 | two weeks; 160 mg once weekly, 40 mg once |
| | weekly; Duration: 52 weeks |
| Placebo Comparator: Placebo every two | Normal saline; |
| weeks; subcutaneous injection; Cohort 1 | Dosage form: Solution for injection; Dosage |
| | and frequency: Isotonic saline solution (0.9% |
| | sodium chloride), once every two weeks/once |
| | every week; Duration: 52 weeks |
| Active Comparator: izokibep 160 mg once | Izokibep 160 mg once every two weeks, 160 |
| every week; subcutaneous injection; | mg once every four weeks, 40 mg once every |
| Cohort 2 | two weeks; 160 mg once weekly, 40 mg once |
| | weekly; Duration: 52 weeks |
| Active Comparator: izokibep 40 mg once | Izokibep 160 mg once every two weeks, 160 |
| every week; subcutaneous injection; | mg once every four weeks, 40 mg once every |
| Cohort 2 | two weeks; 160 mg once weekly, 40 mg once |
| | weekly; Duration: 52 weeks |
| Placebo Comparator: Placebo QW SC | Normal saline; |
| injection; Cohort 2 | Dosage form: Solution for injection; Dosage |
| | and frequency: Isotonic saline solution (0.9% |
| | sodium chloride), once every two weeks/once |
| | every week; Duration: 52 weeks |

### Primary Outcome Measures

1. Proportion of subjects achieving an ASAS40 response [Time Frame: 16 weeks]

### Secondary Outcome Measures

2. Change from baseline in BASDAI [Time Frame: 16 weeks]
3. Change from baseline in BASFI [Time Frame: 16 weeks]
4. Proportion of subjects reaching ASDAS-MI [Time Frame: 16 weeks]
5. Incidence of AEs [Time Frame: 74 weeks]
6. Incidence of serious adverse events (SAEs) [Time Frame: 74 weeks]
7. AEs leading to withdrawal from investigational medicinal product (IMP) [Time Frame: 74 weeks]
8. Proportion of subjects achieving an ASAS40 response at Week 2, 24 and 52 [Time Frame: 52 weeks]
9. Change from baseline in BASDAI at Week 2, 24 and 52 [Time Frame: 52 weeks]
10. Change from baseline in BASFI at Week 2, 24 and 52 [Time Frame: 52 weeks]
11. Proportion of subjects reaching ASDAS-MI at Week 24 and 52 [Time Frame: 52 weeks]

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for treatment of ankylosing spondylitis: the method comprising administering to a patient a therapeutically effective amount of pharmaceutical composition comprising a bispecific fusion protein, wherein the bispecific fusion protein comprises an IL-17A binding motif and an albumin binding motif.

2. A method claim 1, wherein the IL-17A binding motif consists of an amino acid sequence selected from:
(i)
(SEQ ID NO. 1)
EX₂DX4AX₆X7EIX₁₀X11LPNLX16X17X18QX₂₀X21AFIX25X26L
X₂₈X29
wherein, independently from each other,
X₂ is selected from A, H, M and Y;
X₄ is selected from A, D, E, F, K, L, M, N, Q, R, S and Y;
X₆ is selected from A, Q and W;
X₇ is selected from F, I, L, M, V, W and Y;
X₁₀ is selected from A and W;
X₁₁ is selected from A, D, E, F, G, L, M, N, Q, S, T and Y;
X₁₆ is selected from N and T;
X₁₇ is selected from H, W and Y;
X₁₈ is selected from A, D, E, H and V;
X₂₀ is selected from A, G, Q, S and W;
X₂₁ is selected from A, D, E, F, H, K, N, R, T, V, W and Y;
X₂₅ is selected from A, D, E, G, H, I, L, M, N, Q, R, S, T and V;
X₂₆ is selected from K and S;
X₂₈ is selected from I, L, N and R; and
X₂₉ is selected from D and R; and
(ii) an amino acid sequence which has at least 96% identity to the sequence defined in (i).

3. The method of claim 2, wherein the bispecific fusion protein is izokibep.

4. The method of claim 1, wherein the pharmaceutical composition comprises at least one additional excipient.

5. The method of claim 1, wherein the pharmaceutical composition is a solution.

6. The method of claim 5, wherein the solution is an injectable solution.

7. The method of claim 6, wherein the injectable solution is administered subcutaneously.

8. The method of claim 1, wherein the pharmaceutical composition is administered once a week.

9. (canceled)

10. The method of claim 1, wherein the pharmaceutical composition is administered once every two weeks.

11.-14. (canceled)

15. The method of claim 1, wherein the pharmaceutical composition comprises about 160 mg izokibep.

16.-19. (canceled)

20. The method of claim 3, wherein the patient is administered the pharmaceutical composition comprising 160 mg izokibep once every two weeks.

21.-24. (canceled)

25. The method of claim 1, wherein the patient is suffering from axial spondyloarthritis (AxSpA).

26. (canceled)

27. The method of claim 1, wherein the patient is suffering from non-radiographic axial spondyloarthritis (nr-AxSpA).

28. A peptide comprising an IL-17A binding motif and an albumin binding motif for use in the treatment of ankylosing spondylitis (AS), wherein said treatment is as defined in claims 1.

29.-35. (canceled)


SEQUENCE LISTING (3 sequences)

SEQ ID NO: 1 - protein, 29 aa, synthetic construct
  VARIANT 2: note = Xaa is selected from A, H, M and Y
  VARIANT 4: note = Xaa is selected from A, D, E, F, K, L, M, N, Q, R, S and Y
  VARIANT 6: note = Xaa selected from A, G, Q, S and W
  VARIANT 7: note = Xaa is selected from F, I, L, M, V, W and Y
  VARIANT 10: note = Xaa is selected from A and W
  VARIANT 11: note = Xaa is selected from A, D, E, F, G, L, M, N, Q, S, T and Y
  VARIANT 16: note = Xaa is selected from N and T
  VARIANT 17: note = Xaa is selected from H, W and Y
  VARIANT 18: note = Xaa is selected from A, D, E, H and V
  VARIANT 20: note = Xaa selected from A, G, Q, S and W
  VARIANT 21: note = Xaa is selected from A, D, E, F, H, K, N, R, T, V, W and Y
  VARIANT 25: note = Xaa is selected from A, D, E, G, H, I, L, M, N, Q, R, S, T and V
  VARIANT 26: note = Xaa is selected from K and S
  VARIANT 28: note = Xaa is selected from I, L, N and R
  VARIANT 29: note = Xaa is selected from D and R
EXDXAXXEIXXLPNLXXXQXXAFIXXLXX

SEQ ID NO: 2 - protein, 46 aa, synthetic construct
  VARIANT 3: note = Xaa is selected from E, S, Q and C
  VARIANT 6: note = Xaa is selected from E, S and C
  VARIANT 7: note = Xaa is A
  VARIANT 10: note = Xaa is selected from A, S and R
  VARIANT 14: note = Xaa is selected from A, S, C and K
  VARIANT 26: note = Xaa is selected from D and E
  VARIANT 39: note = Xaa is selected from D and E
  VARIANT 40: note = Xaa is A
  VARIANT 43: note = Xaa is selected from A and K
  VARIANT 44: note = Xaa is selected from A and S
  VARIANT 46: note = Xaa is selected from P and -
LAXAKXXANXELDXYGVSDFYKRLIXKAKTVEGVEALKXXILXXLP

SEQ ID NO: 3 - protein, 173 aa, Synthetic construct
AEAKYAKEADDAAVEIASLPNLTWDQWYAFIQKLRDDPSQSSELLSEAKKLNDSQAPKASGSLAEAKEAANAELDSYGVSDFYKRLIDKAKTVEGVEALKDAILAALPGTGGGGSAEAKYAKEADDAAVEIASLPNLTWDQWYAFIQKLRDDPSQSSELLSEAKKLNDSQAPK